(19) United States
(12) Patent Application Publication
(10) Pub. No.: US 2020/0146995 A1
(43) Pub. Date: May 14, 2020

(54) MAGNETIC NANOPARTICLES FOR TARGETED DELIVERY

(71) Applicant: OTOMAGNETICS, INC., Rockville, MD (US)

(72) Inventors: Benjamin Shapiro, Washington, DC (US); Mohammed Shukoor, Washington, DC (US)

(21) Appl. No.: 16/627,677

(22) PCT Filed: Jun. 30, 2018
(86) PCT No.: PCT/US2018/040523

§ 371 Date: Dec. 30, 2019

Related U.S. Application Data
(60) Provisional application No. 62/527,274, filed on Jun. 30, 2017.

(51) Int. Cl. A61K 9/50; A61K 9/51; A61K 31/496; A61K 31/58; A61K 31/573; A61K 9/19

(52) U.S. Cl. CPC A61K 9/5094; A61K 9/5107; A61K 9/5153; A61K 9/5146; A61K 31/58; A61K 31/573; A61K 9/19; A61K 31/496

(57) ABSTRACT

A nanoparticle capable of crossing tissue has an iron oxide core, a first therapeutic agent, and a polymeric coating. The nanoparticles can be sterilized or part of a lyophilized formation.

### CROSS-REFERENCE TO RELATED APPLICATIONS

This application claims priority to and the benefit of U.S. Provisional Patent Application No. 62/527,274, filed Jun. 30, 2017, the entire disclosure of which is hereby incorporated herein by reference.

### FIELD OF THE INVENTION

This application generally relates to targeted drug delivery using therapeutic magnetic particles. More specifically, this application relates to modified ferromagnetic nanoparticles formulated with agents and targeted by magnetic devices.

### BACKGROUND

Nanoparticles are emerging as a new class of therapeutics because they can perform in ways that other therapeutic modalities cannot. Although there are many types of nanoparticles, few will have the proper attributes to reach clinical use because of the issues involved in translating research grade nanoparticles to clinic grade nanoparticles.

Many of the previously disclosed magnetic nanoparticles do not have a geometry, configuration, particle size, or iron-oxide core size, a distribution of core size, and charge and coating elasticity to allow safe and effective movement through tissue barriers to the targets. Many of the previously disclosed particles also do not have the necessary stability, sterility, shelf-life, or ability to carry multiple drugs or other therapeutic payloads.

Prior disclosed magnetic nanoparticles generally have been intended for injection into the body or body part. For example, Asmatulu et al. (US 2012/0265001A1) teaches that magnetic particles must be placed at the site of disease by invasive injection with a syringe, and also teaches the need for a biological targeting agent (e.g. human serum albumin) to effectively reach disease (e.g. cancer) targets by the mechanism of tumors uptaking albumin to support their metabolism. Such techniques can disperse agents for the iron-oxide cores. These techniques are not suitable for passing through tissue.

Accordingly, there is always a need for improved nanoparticles. There is a need for magnetic nanoparticles that can cross tissue barriers in response to a magnetic gradient (e.g., from a site of deposition) and that can effectively deliver without biological targeting agents (e.g., albumin, antibody, gene, nucleotide or other targeting agents). It is to these needs, among others, that this disclosure is directed.

### SUMMARY

One aspect includes nanoparticles that can deliver therapies or multiple therapies across tissue barriers to targets behind them. These nanoparticles include a carrier with pores and therapeutic agent(s) smaller than the pores. For example, nanoparticle can deliver large molecules (large molecule therapies, proteins, antibodies, nucleotides or gene therapy) through tissue barriers to targets. Such large molecules are often too large to cross tissue barriers by diffusion, and the nanoparticles can transport them across tissue barriers in response or with the action of an applied magnetic gradient.

Another aspect is nanoparticles loaded with multiple drugs or therapies, thus enabling delivery of more than one agents to a target site.

Another aspect includes particles or nanoparticles having magnetic or superparamagnetic iron oxide cores (e.g. magnetite, maghemite, or other iron oxides) inside the polymeric coating or matrix. Iron is naturally found in the human body, and iron is readily absorbed by the body for use in red blood cells. These nanoparticles can be bio-compatible, and in exemplary particles only contain materials previously approved by the FDA as safe for injection into the human body.

Another aspect includes nanoparticles that can effectively move through or across tissue barriers by an applied magnetic gradient. Generally, tissue keeps materials out. For example, the epithelium of the skin prevents entry of materials through the skin and into the body, or the external sclera of the eye prevents materials from entering the eye. Other tissue barriers are seen in the ear drum, the window membranes, membranes between or that surround organs, liquid barriers (such as the vitreous of the eye, or effusion that fills or partially fills the middle ear during otitis media with effusion), or tissue barriers due to muscle, fat, bone or other tissue types.

Another aspect includes compositions or pharmaceutical compositions having particles that are substantially mono-dispersed or have a narrow particle size distribution. In exemplary particles, the iron oxide cores are mono-dispersed, have a narrow size distribution.

Another aspect includes nanoparticles with biodegradable (e.g., in water at about 37 degrees) polymeric coating and which are capable of holding multiple biologically active agents. In one example, the coating may include PLGA and allows for multiple therapies/therapeutic agents (e.g. loading with hydrophobic, hydrophilic, and lipophilic molecules). There can be multiple therapies at the same time (e.g. with an antibiotic and an anti-inflammatory) and allows sequential release of therapies. This enabling on-demand timed release of one or multiple different therapies. This method allows simultaneous encapsulation of two or more drugs with different chemical signatures, such as, solubility (hydrophilic and hydrophobic), charge (cationic, anionic, and/or zwitterion), pH-dependence, lipophilicity, etc. into a single nanoparticle. In some examples, the drugs are not chemically altered and/or conjugated with other reagents and are loaded in their native form.

Another aspect nanoparticles with multiple agents and method for loading nanoparticles with multiple agents. Certain examples include agents with agent having disparate pKa values. Such zwitterionic drugs exhibit solubility for a wider pH range and often results in low encapsulation efficiency due to leakage. In one example, a pH-dependent solubility of ciprofloxacin was reduced/inhibited by formation of a Hydrophobic Ion Complex (HIP) between the drug of interest and a surfactant. Steroids on the other hand are highly hydrophobic and exhibits minimal-to-no aqueous solubility. These compounds are otherwise soluble in organic solvents which are often non-biocompatible and poses high health risks. Specific examples include the nanoparticles with medium and large molecular weight drugs and biomolecules.

The degradation rate of the polymeric coating (e.g., PLGA) under physiological conditions and the size of the pores allow for fast ‘burst’ release of the therapy (in minutes or hours) or slow sustained release of the therapy (over weeks or months). The polymer and the agent can be selected to treat a specific disease targets (e.g. a faster profile to quickly kill an infection, or slower profile to provide sustained treatment for a chronic or long-lasting condition).

Another aspect includes nanoparticles with a varying range of particle size. The size of the particles can be from 10 nm to 450 nm diameter, the size of the internal iron oxide cores may be from 1 nm to 50 nm. The iron content (5-40%) has been selected to maximize delivery of therapy through tissue barriers to the targets behind them.

Another aspect includes nanoparticles or compositions thereof that are sterile. Sterility is achieved either by gamma or e-beam irradiation or by filtration.

Another aspect includes pharmaceutical formulation or compositions of nanoparticles that has a longer shelf life achieved by lyophilization (freeze drying). The particle and therapy formulation can be safely stored on a shelf and then reconstituted by adding water or saline or other buffer immediately before use.

Another aspect includes nanoparticles that can be contained in an aqueous buffer solution. For situations where this solution is first placed in a non-aqueous environment (e.g. on the surface of oily skin) before a magnetic field is applied, for those situations effective surfactants (such as exemplary surfactants cetrimonium chloride, sodium lauryl sulphate, poloxamer, Triton X-100, carboxymethylcellulose sodium, polysorbates (20, 40, 60, 80), benzyl alcohol, etc. which were previously approved for use by the FDA) can be included in the buffer that contains the particles. This reduces the surface tension of the buffer and enables the particles to easily leave the buffer, and to enter and then cross the tissue barrier (e.g. to readily enter and cross oily skin). Exemplary surfactants or other additives may also allow improved transport through tissue barriers by other means that are recognized in the field, e.g. by improved interactions with surface charge of cells and tissues, by modifying tight cell junctions, or by enabling better transport between cells and through membrane networks. Another reason to add surfactants or other chemicals into the liquid around the particles is to modify the strength of the tissue barriers (e.g. to reduce the strength of tight junctions between barrier cells).

According to a still further aspect, a kit is provided, comprising a nanoparticle according to this disclosure.

Further aspects and embodiments of the invention will be apparent from the following description and the appended claims.

### BRIEF DESCRIPTION OF THE DRAWINGS

FIG. 1 shows magnetic nanoparticles schematically traveling through tissue and delivering a therapy (drugs, proteins, nucleotides) behind or across the tissue barrier.

FIG. 2A shows an exemplary design for a nanoparticle system consisting of a single Fe2O3 or Fe3O4 core, coated with small molecule ligands, polymeric ligands such as PEG and or block copolymers.

FIG. 2B shows another exemplary design for an unfunctionalized PLGA magnetic nanoparticle

FIG. 2C show another exemplary design for an unfunctionalized PLGA magnetic nanoparticle for transporting agents through tissue barriers.

FIG. 2D shows another exemplary design for a cationic PLGA nanoparticle loaded with drug PSA.

FIG. 2E shows another design for a cationic PLGA nanoparticle loaded with drug PSA.

FIG. 2F shows another exemplary design for a cationic PLGA nanoparticle loaded with drug PSA.

FIG. 2G another schematic design for cationic PLGA nanoparticle encapsulating PSA.

FIG. 3A shows exemplary magnetic PLG coated nanoparticles with 5 nm iron oxide cores traverse tissue barriers

FIG. 3B shows exemplary PLGA coated magnetic nanoparticles with 10 nm iron oxide cores able to traverse tissue barriers

FIG. 3C shows PLGA coated magnetic nanoparticles with 20 nm iron oxide cores capable of crossing tissue barriers.

FIG. 4 shows that the exemplary nanoparticles on glass slide in aqueous buffer.

FIGS. 5A through 5C show the results from image processing to determine particle speed through media.

FIG. 6 shows a schematic view of a manufacturing process of an exemplary nanoparticle.

FIG. 7A shows Prussian staining of iron oxide after delivery into cow eyes and verified that examplary PLGA iron-oxide nano-particles could traverse the epithelial layer of the eye.

FIG.7B shows Prussian staining of iron oxide after delivery into cow eyes and verified that examplary PLGA iron-oxide nano-particles could traverse the epithelial layer of the eye.

### DETAILED DESCRIPTION

Nanoparticle formulations for delivering multiple therapeutic agents are disclosed. Specific embodiments include magnetic nanoparticles having a single therapeutic agent or multiple therapeutic agents. These particles may have at least one dimension of about 3 nanometers, about 10 nanometers, 100 nanometers or more. Such magnetic nanoparticles can offer medical treatment options by manipulating their movement using an externally applied magnetic field gradient, more specifically by having the particle traverse (cross) intact tissue barriers under the action of a magnetic field. Certain nanoparticles can be used in a therapeutic and/or diagnostic clinical procedure.

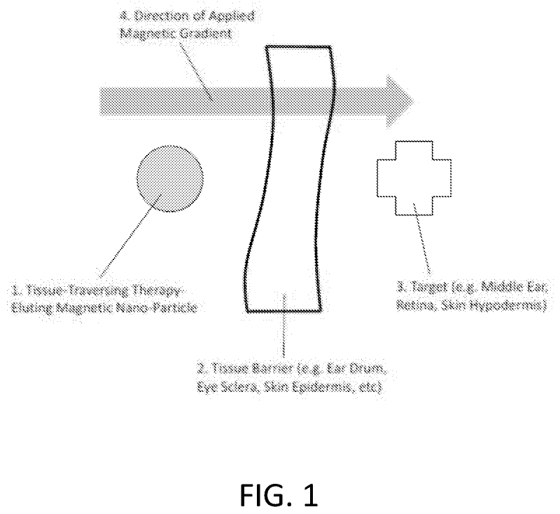

FIG. 1 shows schematically magnetic nanoparticles traveling through or accross tissue barriers to deliver therapy (drugs, proteins, nucleotides) at disease targets behind those tissue barriers. This figure shows a therapy-eluting nano-particles that can traverse tissue barriers under the action of an applied magnetic gradient.

In embodiment, the nanoparticle capable of crosses tissue has an iron oxide core (e.g., singular core or multicore) a first therapeutic agent, and a polymeric coating or matrix, wherein degrades in water at about 37 degrees.

One example includes a PLGA (poly lactic-co-glycolic acid) nanoparticles, with iron-oxide nano-cores. The nanoparticle can be loaded with a therapeutic agent in the polymer matrix (PLGA or PEG or poloxamer nonionic triblock copolymers composed of a central hydrophobic chain of (poly(propylene oxide)) flanked by two hydrophilic chains of polyoxyethylene(poly(ethylene oxide) (or polycaprolactone or povidone, etc.) stabilized by PVA (polyvinyl alcohol) and/or chitosan and lyophilized (flash frozen).

In one embodiment, the nanoparticles may be filtered or gamma or e-beam irradiated for sterility. The particles generally consist of one or many magnetic cores (magnetite Fe₃O₄, maghemite γ-Fe₂O₃, and/or other iron oxidation products) and a surrounding polymer matrix. In one example, the cores may be magnetite or maghemite, which are naturally occurring iron oxides. In one example, the nanoparticles have a neutral, surface charge, single iron oxide core, are relatively stiff, have a size between about 5-50 nm or 30-250 nm are lyophilized, are sterilized. A wide range of polymer-based coating or matrix materials are used (PEG, hyaluronate, poloxamers, etc.) to (a) encapsulate drug/s and further render nanoparticles—cationic, hydrophilic, anionic, etc. For biocompatibility, and biodegradablility and therapy release profiles and rates, the polymer/s can be custom selected based on molecular weight, density, and functional end groups. The nanoparticle may have a polydispersity index (PDI) of between about 0.1-0.5. That means the nanoparticles distribution is homogenous with little size variance or particle heterogeneity.

In another example, the nanoparticles have a positive surface charge, have multiple cores, are relatively stiff, have a size between about 10-400 or 180-350 nm (nanometers), are lyophilized, are sterilized. In another examples, nanoparticles can be primarily composed of the polymer PLGA (polylactic-co-glycolic acid). In exemplary particles, the PLGA may have L:G=50:50 and Molecular weight (Mw)=30 kDa-50 kDa. In other examples, the PLGA molecular weight range varied from 10 kDa to 100 kDa. PLGA can have the functional end groups—carboxylic, -amine, -ester. The lactide:galactide can have a ratio varying (50:50, 65:35, 75:25, 85:15).

In another embodiment, the nanoparticles may be lyophilized in the presence of sugar (e.g. trehalose, mannitol, sucrose, or glucose). That leads to the nanoparticles being coated with sugar in their lyophilized state. For biocompatibility, and biodegradablility and therapy release profiles and rates, the particle PLGA can be tuned by choosing a molecular weight, a compositional ratio (e.g., lactide to galactide), a density, and functional end groups. The nanoparticle may have a polydispersity index (PDI) of between about 0.1-0.5. That means the nanoparticles distribution is homogenous with little size variance or particle heterogeneity.

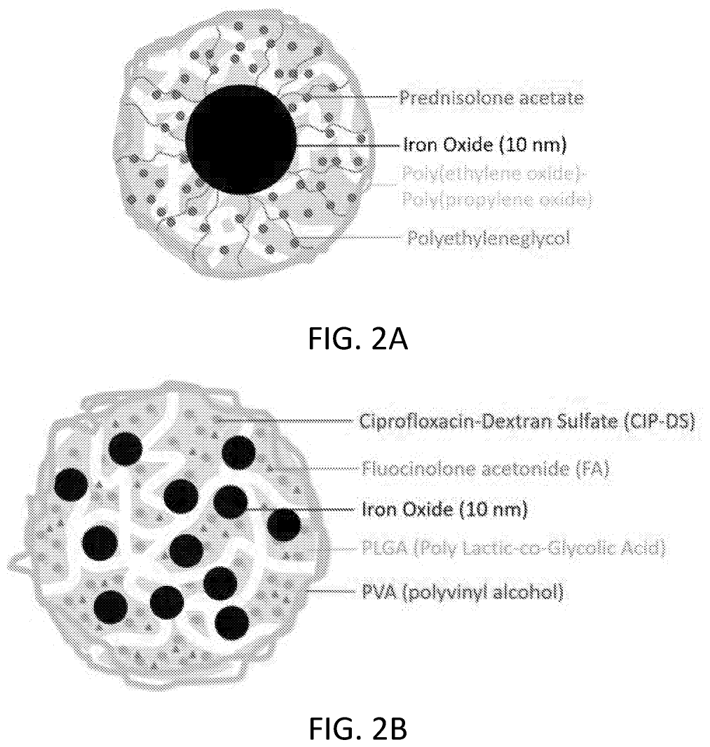

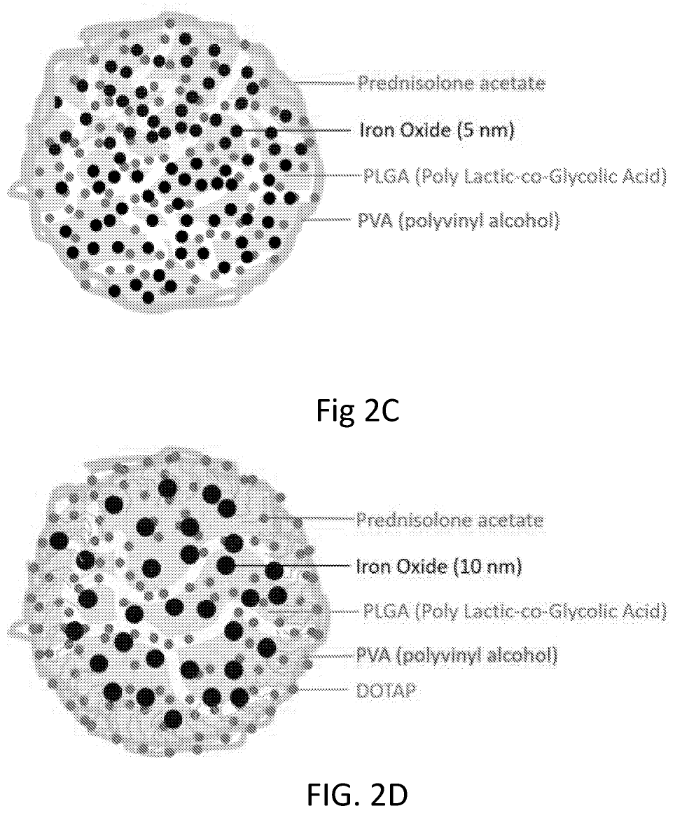

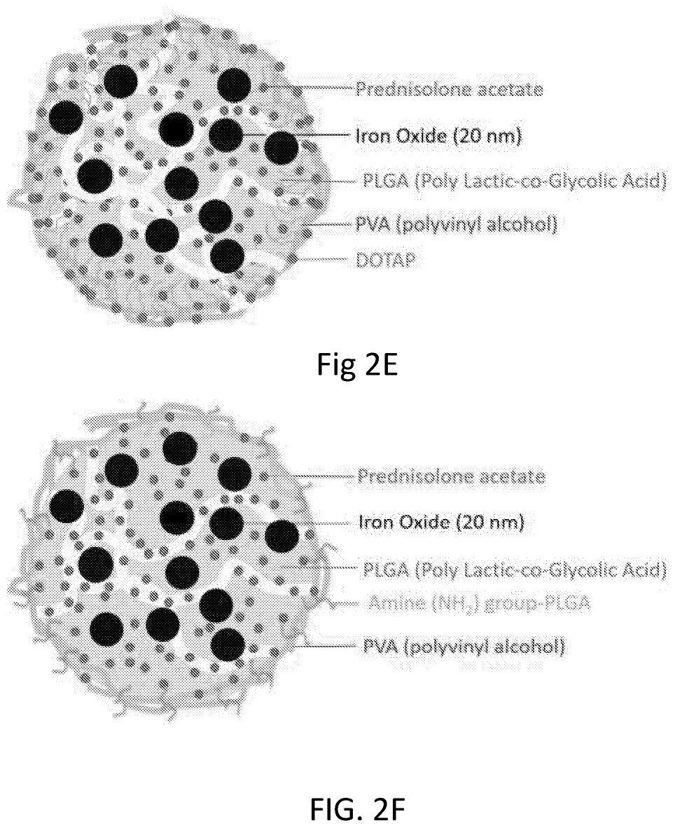

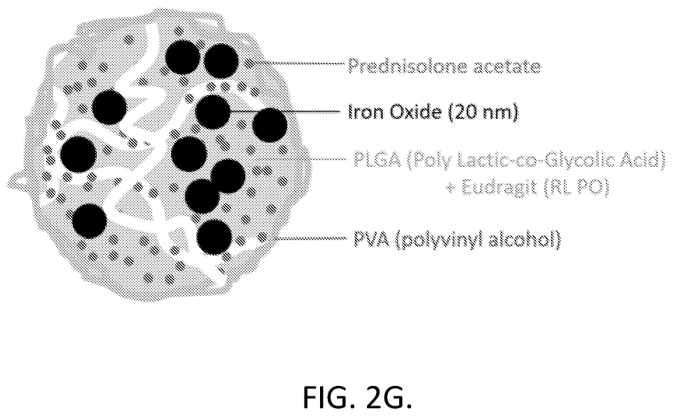

FIGS. 2A, 2B, 2C, 2D, 2E, and 2F show examples of magnetic nanoparticles able to traverse tissue barriers under the action of a magnetic gradient, and able to carry and deliver therapy to the targets behind those barriers.

FIG. 2A shows another schematic design for a nanoparticle system consisting of a single Fe₂O₃ or Fe₃O₄ core, coated with small molecule ligands, polymeric ligands such as PEG and or block copolymers such as Poloxamers (F68, F127, etc.) encapsulating single or multiple drugs and validated for transport through tissue barriers under the action of a magnetic gradient. The agents can be either premixed with the iron oxide cores before coating or matrix with polymeric ligands or can be simultaneously loaded while the coating or matrix the iron oxide cores in a single step. For the current system the size of the iron oxide core ranges between 5 and 30 nm. The composition, features, and properties of this particle have been selected, based on the concepts disclosed herein, to allow delivery of therapy through tissue barriers to the targets behind them. The PLGA matrix can be also be loaded with a variety of therapies, with small or large molecule drugs, with proteins or antibodies, or with nucleotides (genes, DNA, RNA, mRNA, siRNA, etc).

FIG. 2B shows another schematic design for an unfunctionalized PLGA magnetic nanoparticle for transport through tissue barriers under the action of a magnetic gradient. The PLGA nanoparticle is negatively charged and is co-loaded with more than one drug or therapies with different chemical signatures (solubility, hydrophilicity and hydrophobicity, charge (cationic, anionic, and/or zwitterion), pH-dependence, lipophilicity, etc.). As can be seen, two different class drugs, e.g., (1) zwitterionic antibiotic (Ciprofloxacin) and (2) lipohilic/hydrophobic steroid (Fluocinolone acetonide) are co-loaded into a single nanoparticle. Ciprofloxacin is soluble in a wide pH range (acidic pKa1=6.2 and basic pKa2=8.8), this pH-dependent solubility of ciprofloxacin was reduced/inhibited by formation of a Hydrophobic Ion Complex (HIP) between ciprofloxacin and surfactant, Dextran sulfate. The complex is introduced into the nanoparticle along with fluocinolone acetonide and magnetic iron oxide cores (10 nm). The PLGA matrix can be also be loaded with a variety of therapies, with one or more agents, with small or large molecule drugs, with proteins or antibodies, or with nucleotides (genes, DNA, RNA, mRNA, siRNA, etc).

FIG. 2C another schematic design for an unfunctionalized PLGA magnetic nanoparticle for transporting agents through tissue barriers under the action of a magnetic gradient. The PLGA nanoparticle is negatively charged and loaded with drug PSA (prednisolone acetate) and magnetic iron oxide cores (5 nm). to allow delivery of therapy through tissue barriers to the targets behind them. The PLGA matrix can be also be loaded with a variety of therapies, with small or large molecule drugs, with proteins or antibodies, or with nucleotides.

FIG. 2D shows another schematic design design for a cationic PLGA nanoparticle loaded with drug PSA and magnetic iron oxide cores (10 nm). The nanoparticle incorporates a cationic phospholipid, N-[1-(2,3-Dioleoyloxy)propyl]-N,N,N-trimethylammonium methyl-sulfate (DOTAP), to render positive surface charge. The pores in the PLGA can be loaded with a variety of therapies, with one ore more agents, with small or large molecule drugs, with proteins or antibodies, or with nucleotides.

FIG. 2E shows another schematic design for a cationic PLGA nanoparticle loaded with drug PSA and magnetic iron oxide cores (20 nm). The nanoparticle incorporates a cationic phospholipid, DOTAP, to render positive surface charge. The pores in the PLGA can be loaded with a variety of therapies, with one or multiple therapies, with small or large molecule drugs, with proteins or antibodies, or with nucleotides (genes, DNA, RNA, mRNA, siRNA, etc).

FIG. 2F shows another schematic design for a cationic PLGA nanoparticle loaded with drug PSA and magnetic iron oxide core or cores (20 nm). The nanoparticle is made of PLGA with amine (NH₂) functional groups (PLGA-NH₂) to render positive surface charge. The pores in the PLGA can be loaded with a variety of therapies, with small or large molecule drugs, with proteins or antibodies, or with nucleotides (genes, DNA, RNA, mRNA, siRNA, etc).

FIG. 2G another schematic design for cationic PLGA nanoparticle encapsulating PSA and magnetic iron oxide cores (20 nm). The nanoparticle matrix is a blend of PLGA and Eudragit (RL PO) polymers containing amine (NH₂) end groups to render positive surface charge. The pores in the PLGA can be loaded with a variety of therapies, with small or large molecule drugs, with proteins or antibodies, or with nucleotides (genes, DNA, RNA, mRNA, siRNA, etc)

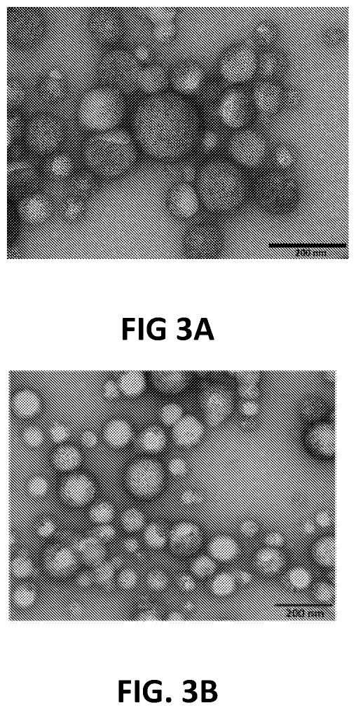

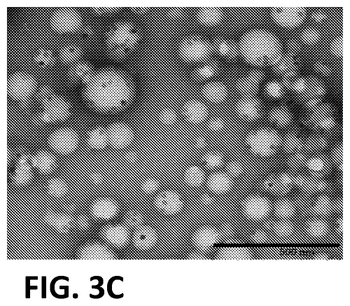

FIG. 3A-3C show TEM (Transmission Electron Microscope) images showing PLGA nanoparticles loaded with iron oxide cores and also provides a measure of particle size (see particle size versus scale bar). FIG. 3A shows exemplary magnetic PLG coated nanoparticles with 5 nm iron oxide cores traverse tissue barriers under the action of a magnetic gradient and able to carry and deliver therapy to the targets behind those barriers. FIG. 3B shows PLGA coated magnetic nanoparticles with 10 nm iron oxide cores able to traverse tissue barriers under the action of a magnetic gradient, and able to carry and deliver agents to the targets behind those barriers. FIG. 3C shows PLGA coated magnetic nanoparticles with 20 nm iron oxide cores capable of crossing tissue barriers under the action of a magnetic gradient, and able to carry and deliver agents to the targets behind those barriers. The TEM image shows provides a measure of particle size (see particle size versus scale bar).

The method provides mono-dispersity, a narrow size distribution, both for the particles and for the iron-oxide cores inside the particles. In one exemplary instance, our particles are made with a narrow size distribution of 200-250 nm (nanometers) in diameter. In other exemplary instances the particles are smaller, with size ranges between 20-50 nm or 20-100 nm.

In one embodiment, the nanoparticles may contain a pharmaceutical agent. The agent may be a drug, a protein, or nucleotide material (e.g., DNA, mRNA, siRNA). The magnetic particles may take various forms. A magnetic particle may comprise magnetic cores and a matrix in which the therapeutic agent is contained.

The pharmaceutical agent may include DNA, RNA, interfering RNA (RNAi), siRNA, a peptide, polypeptide, an aptamer, a drug, a small or a large molecule. Small molecules may include, but are not limited to, proteins, peptides, peptidomimetics (e.g., peptoids), drugs, steroids, antibiotics, amino acids, polynucleotides, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The therapeutic agent may comprise a therapeutic agent for preventing or treating an ear or eye or skin disease or injury, and the target location may comprise ear or eye tissue or tissues in or underneath the skin. The therapeutic agent may comprise a steroid, e.g. an anti-inflammatory steroid, for delivery to the inner ear (cochlea and/or vestibular system) as a target location, to treat conditions such as hearing loss, tinnitus, vertigo, Meniere's, to protect hearing from chemotherapy regimens or from other medications that damage hearing (e.g. loop diuretics, some antibiotics such as aminoglycosides, non-steroidal anti-inflammatory drugs, etc.), and to treat other conditions of the inner ear. The therapeutic agent may also include drugs, proteins, growth factors (including stem cell derived factors), or nucleotides or genes, for delivery to the inner ear, for example to protect, recover, or restore hearing (e.g. by delivering growth factors to cause cochlear hair cells and support cells to grow and thereby restore hearing, or by delivering nucleotides or genes that would cause the body to initiate cochlear hair cell and support cell growth). Therapeutic agents may include prednisolone, dexamethasone, STS (sodium thiosulfate), D-Methionine, Triamcinolone Acetonide, CHCP 1 or 2, epigallocatechin gallate (EGCG), Glutathione, Glutathione reductase, and others. For delivery of the particles plus therapeutic agent to the inner ear, the particles would traverse (cross) intact oval and/or the round window membranes under the action of a magnetic field to deliver the therapy to the inner ear.

The therapeutic agent may comprise anti-inflammatory steroids and antibiotics, for delivery to the middle ear as a target location, to treat conditions such as middle ear infections and inflammations (otitis media). The therapeutic agent may include ciprofloxacin and fluocinolone acetonide or ciprofloxacin and dexamethasone. The therapeutic agent may also include drugs, proteins, nucleotides or genes, or other agents, to treat middle ear infections and inflammations. For delivery of the particles plus therapeutic agent to the middle ear, the particles would traverse (cross) the ear drum (tympanic membrane) under the action of a magnetic field to deliver the therapy to the middle ear. Such traversal and therapy delivery does not require that the ear drum (tympanic membrane) be open, that it be surgically punctured or accidentally ruptured.

In one example, the therapeutic agent can have a coating or matrix (e.g. chitosan) based on tissue properties, e.g. for mucolytic, mucoadhesive, other. By introducing a charge or lack of charge (e.g., cationic, anionic and neutral) on nanoparticles surface using single molecule ligands, oligomers, bio/polymers (covering a wide range of molecular weights (100 Da-300,000 Da), the ease at which the particle may travel can be controlled. Other coatings such as a hydrophilic coating of nanoparticles using Pluronics (F127, F68, etc.) or PEGylation of nanoparticles using polyethylene glycol (PEG) for muco-inert nanoparticles may also control such properties.

In one examples, the nanoparticles have modifications for emulsion polymerization. This includes the introduction of co-solvents to reduce nanoparticle size and Solid/oil/water emulsions. Using surfactants/lipids as emulsion stabilizers at oil/water interface.

Depending upon the nature of the molecules to be encapsulated, a wide choice of preparations is available such as desolvation, heat denaturation, coacervation, cross-linking, nano precipitation emulsification, etc. The particle size of the system can be fine-tuned with slight changes in synthesis parameters such as temperature, pH, etc. Moreover, the nanoparticles possess greater stability during storage or in vivo after administration and provide surface functional groups for conjugation to cancer targeting ligands. They also are suitable for administration through different routes.

Any surfactant can be used in the nanoparticles and production methods of the application, including, for example, one or more anionic, cationic, non-ionic (neutral), and/or Zwitterionic surfactants. Examples of anionic surfactants include, but are not limited to, sodium dodecyl sulfate (SDS), ammonium lauryl sulfate, other alkyl sulfate salts, sodium laureth sulfate (also known as sodium lauryl ether sulfate: SLES), or Alkyl benzene sulfonate. Examples of cationic surfactant include, but are not limited to, alkyltrimethylammonium salts, cetylpyridinium chloride (CPC), polyethoxylated tallow amine (POEA), benzalkonium chloride (BAC), and benzethonium chloride (BZT). Examples of Zwitterionic surfactant include, but are not limited to, dodecyl betaine, dodecyl dimethylamine oxide, cocamidopropyl betaine, and coco ampho glycinate. Examples of nonionic surfactant include, but are not limited to, alkyl poly(ethylene oxide), or alkyl polyglucosides (octyl glucoside and decyl maltoside). Examples of non-ionic surfactants include, but are not limited to, polyglycerol alkyl ethers, glucosyl dialkyl ethers, crownethers, ester-linked surfactants, polyoxyethylene alkyl ethers, Brij, Spans (sorbitan esters) and Tweens (Polysorbates).

In embodiment, the nanoparticle includes Hydrophobic Ion Pairing (HIP) within the nanoparticle. HIP complex formation between charged/zwitterionic drugs, proteins, biomolecules (RNA, DNA, etc.) and surfactants (including lipids, polymers, single molecules). HIP complexation surfactants: SDS, docusate sodium, sodium deoxycholate, dextran sulfate, etc. One (in-situ) step HIP complexation. Two (post-modification) steps HIP complexation. For example, a method for preparing a nanoparticle composition has a first agent and a second agent, comprising forming a hydrophobic ion complex between the first agent and adding the second agent after the formation of the hydrophobic ion complex.

The morphology of the nanoparticle can vary. In some examples, the nanoparticles may be a single magnetic (Fe2O3/Fe3O4 core-PLGA shell). In other examples, the nanoparticles may be a multi-cores cluster magnetic (Fe2O3/Fe3O4-PLGA shell). In other the nanoparticles may be Chitosan or Pluronics (F68, F127) or PEG coated Fe2O3/Fe3O4 cores.

The composition may contain excipients. Such excipients includes stablizers, chemical permeation enhancers, preservatives, antimicrobial agents, and pH stablizers. Examplary stabilizers to enhance nanoparticle dispersibility and to reduce/limit nanoparticle aggregation or precipitation upon reformulation in buffer. Ionic, non-ionic (steric), single molecule, polymer-base excipients may be used. Chemical permeation enhancers to enhance/promote nanoparticle penetration/movement through tissue barrier and reversibly. Small molecules: solvents, fatty acids, surfactants, terpenes, etc. Macromolecule-based: polymers, biopolymers, single molecule ligands may also be added.

In embodiment, the nanoparticles include the loading or coloading of active agents. For examples, the nanoparticles can be loaded with fluocinolone acetonide onto nanoparticles, Co-loading ciprofloxacin and fluocinolone acetonide onto HNPs., Co-loading ciprofloxacin and dexamethasone onto nanoparticles. In one examples, the nanoparticles contain a therapeutic agent selected from ciprofloxacin, fluocinolone acetonide or dexamethasone. In another example, the nanoparticles contain two or more therapeutic agents selected from the group consisting of ciprofloxacin, fluocinolone acetonide, dexamethasone or combinations thereof. In yet another examples, the nanoparticles contain ciprofloxacin, fluocinolone acetonide, dexamethasone. The dosage or ratios may be varied extensively (e.g., single vs multiple doses.)

The drug release profile may show various pharmacokinetics, pharmacodynamics (e.g., fast burst release vs slow sustained). We now further disclose selecting the size of the pores for fast (burst) or slow (sustained) release of therapy. Large pores allow release of therapy faster (burst release); small pores release therapy more slowly (sustained release). We further disclose tuning the bio-degradation rate of the polymer for physiological conditions. By selecting a polymer or PLGA with high density cross-linking, the polymer would degrade slowly in the body and would release therapy slowly. By selecting a polymer or PLGA with low density cross-linking, the polymer or PLGA would degrade quickly and release therapy quickly. A burst release of drug or therapy can be achieved by rendering the PLGA polymer more hydrophilic by increasing the galactide content, by reducing the nanoparticle size by using low molecular weight PLGA, and by coating of nanoparticle surface with hydrophilic stabilizers. A slow and sustained release of drug or therapy can be achieved by resisting the water diffusion rate into nanoparticles by increasing hydrophobicity of PLGA, by reducing or restricting the drug or therapy localization on nanoparticle surface, and by increasing the nanoparticle size. In certain examples, the exemplary particles can release therapy quickly (in hours) or slowly (over weeks or months). A selection PLGA attributes (molecular weight and L:G ratio), drug type (hydrophobic, hydrophilic, or lipophilic), and stabilizer chemistry allows for customization the magnetic particles to achieve burst and/or sustained release of drug.

The therapeutic agent may comprise agents used for treatment of eye conditions, e.g. VEGF (vascular endothelial growth factor) or related compounds for macular degeneration, or drugs or proteins or other therapies used for treatment of glaucoma or other conditions of the eye. For delivery of the particles plus therapeutic agent into various tissues of the eye, under the action of a magnetic field the particles would traverse (cross) the sclera, and/or the corneal epithelium, and/or the vitreous humor, and/or other parts of the eye, to reach target tissues in the eye such as the retina, the eye stroma, the anterior chamber of the eye, or other targets within the eye.

The therapeutic agent may comprise agents used for treatment of skin conditions, for treatment of burns or wounds, or treatment of bed sores or ulcers (including diabetic ulcers), or agents that are used to treat other conditions of the body but that are currently delivered through the skin (e.g. vaccines, Botox, etc). The agent may be drugs, proteins, or nucleotides, or other therapeutic agents. The target location may be deeper layers of the skin, layers of the epidermis, the dermis, the hypodermis, or the underlying tissues or blood vessels. Under the action of a magnetic field, the particles would traverse (cross) layers of the skin, to reach underlying target skin layers or other tissues.

The therapeutic agent may generally be drugs, proteins, factors (e.g. derived from stem or other cells), or nucleotides (genes, DNA, RNA, mRNA, siRNA, etc). Under the action of a magnetic fields, the particles may cross tissue barriers to reach the disease or injury targets behind those barriers and deliver the therapeutic agent or agents.

The particle can be tune for the release rate of the therapy contained inside it. A person knowledgeable in the art of drug delivery will recognize that in some instances a fast ‘burst’ release can be desired (e.g. in minutes or hours), for example to quickly suppress an acute inflammation or to rapidly eliminate an infection. In other instances, a slow or sustained release of therapy can be desired (over weeks or months), for example to offer treatment for chronic conditions or relief in the long-term (such as, for example, treatment of recurrent or chronic middle ear infections or inflammations; protect hearing from long-term chemotherapy regimens; or provide sustained therapy release for persistent conditions of the eye such as macular degeneration or glaucoma). In some situations, it is desirable to have a burst release followed by sustained therapy.

In other cases, it may be desirable to release more than one therapy, together or in sequence. To release multiple therapies together, more than one therapy may be loaded in the pores of our particles. Further, to release therapy in sequence, we disclose hybrid PLGA nanoparticles which provide the possibility to load two different drugs in the same nanoparticle system. For instance, our exemplary PLGA-lipid core-shell hybrid nanoparticles can carry hydrophobic drug within the PLGA core and a more lipophilic drug can be loaded within the bilayers of the surrounding lipids shell.

Specific particles have been invented to enable magnetic delivery of treatment to disease targets behind tissue barriers in patients. There are many factors that must be achieved to enable safe and effective treatment in patients (as noted earlier, to date there is only one magnetic nanoparticle that has been FDA approved to treat patients, and that particle cannot carry any therapy other than the iron oxide cores which make it magnetic and which provide the iron to treat iron-deficiency anemia in patients). Aspects of our particle include achieve the criteria that will allow safe and effective treatment of patients, and is anticipated to enable FDA approval and/or approval by other regulatory agencies.

In particular, to ensure safety in the human body, we selected iron-oxide as the material to make our particles magnetic. In comparison to other materials that are also magnetic and that have been used in magnetic nanoparticles in prior art (cobalt, nickel, aluminum, bismuth), in contrast to these iron oxide is a material naturally found in the human body, it is readily absorbed by the body for use in red blood cells, and the FDA has previously approved iron oxide as safe material for inj ection into the human body.

In a use, a magnetic system can be used to apply a magnetic force to the particles so as to tend to move the particles in directions towards or away from the magnetic system. Specifically, the particles may be moved through tissue barriers to disease or injury targets behind them. Specific examples and embodiment provide iron-oxide nano-particles provide safe and effective magnetic delivery (e.g. magnetic injection) to targets in the body. In one instance, these particles could be loaded with antibiotics and/or anti-inflammatory drugs and placed in the outer ear. A magnetic gradient would then deliver them through the ear drum to the middle ear, to clear middle ear infections and to reduce middle ear inflammation. This would enable treatment of middle ear infections without systemic antibiotics (for acute infections) or without a tympanostomy tube surgery which involves insertion of a tube through an ear drum (typically for treatment of recurrent or chronic middle ear infections and inflammations in children). In another instance, these particles could be placed on the surface of the eye, and then a magnetic gradient could be applied to transport these particles through the sclera to targets inside the eye, e.g. to behind the lens, into the vitreous, or to the retina. This could obviate the need for needle injections into the eye. In yet another instance, these particles could be placed on the skin, and a magnetic gradient could be applied to transport them through the epidermis of the skin to target layers underneath the epidermis. The magnetic gradient could be applied by one or multiple magnets pulling the particles towards them (magnetic gradient towards the magnets), or by a magnetic injection device (magnetic gradient going away from the device). In both cases, the particles would react to the direction of the applied magnetic gradient (e.g., FIG. 1).

The magnetic particles may be formed in any of a number of suitable ways. A particle may be formed by the steps of reagent preparation and mixing, emulsification and solvent evaporation and washing and lyophilization. For example, a particle may be formed with a matrix in which magnetic material is carried as iron-oxide nano-cores and in which the therapeutic agent is also carried. The magnetic particle has a matrix, such as a PLGA polymer matrix, carrying magnetic material as iron-oxide nano-cores. Therapeutic agent can be also carried in the matrix. Such particles can be made in various ways.

In some examples, the diameter of the PLGA nanoparticles is between about 100 to about 400 nm. In another examples, the diameter of are between about 130 to about 400. In yet other examples, the diameter is between about 130 to about 220 nm. In yet other examples, the diameter is between 20 to about 100 nm.

Another embodiment includes a method for creating a sterile nanoparticle formation. The magnetic nano-particle is irradiated by gamma radiation or by e-beam (electron beam) radiation for a dose ranging from 5 kGy to 22 kGy. Such radiation destroys and kills microorganisms and provides a sterile formulation. selection of particle properties (size, polymer, composition) and of the radiation dose, and validating experiments, ensure that any microorganisms are reliably destroyed but the therapy contained inside the particle is not. In a second instance (alternate sterility procedure), the size of the particle is selected to be below 220 nm in diameter, and in some cases below 180 nm in diameter, to increase yield during sterilization filtration. In this second instance, the nano-particles are passed through 0.22 um (220 nm) micron rated filters recommended in FDA guidance documents, to filter out microorganisms and to ensure formulation sterility.

Another embodiment includes a lyophilized formulation, which adds shelf life. Lyophilization, or freeze drying, is a process in which the material is frozen (e.g. −80 C for 24 hours or is flash frozen using liquid nitrogen (N2)) and dried under high vacuum. The nanoparticles are lyophilized in the presence of sugars (e.g. trehalose, mannitol, sucrose, glucose), and cause the nanoparticles to be coated with such sugars during lyophilization. The results are a stable powder that has a long shelf life (e.g. two years or more), including at room temperature conditions. When the lyophilized formulation is stored, therapy (drugs, proteins, or genes) do not exit from or leak out from the particles (the therapy loading remains stable over time). To use, our particles are reconstituted by the addition of water, saline, or buffer. Reconstitution can be achieved in an easy to use vial. In one example, there can be a double chamber vial in which one twists and buffer pours in from top chamber, then the user mixes the vial to reconstitute the formulation. In one example, sugars, polyols, mannitol, and/or sorbitol may be used during the process. Other examples of stabilizers include sucrose, trehalose, mannitol, polyvinylpyrrolidone (PVP), dextrose, and glycine. These agents can be used in combination, such as sucrose and mannitol, to produce both an amorphous and crystalline structure. Another embodiment includes a method for treating a patient, comprising providing a lyophilized composition of nanoparticles, reconstituting the nanoparticles, applying the nanoparticles to a site, and moving the nanoparticles to a target site using a magnetic gradient.

### EXAMPLES

### Example 1

The particles, for safely and effectively traversing tissue barriers under the action of an applied magnetic gradient, are composed of biodegradable and biocompatible materials such as PLGA (in particular, exemplary particles are composed solely of materials previously approved by the FDA for administration into the body). Exemplary nanoparticles exhibit the capability of encapsulating magnetic cores of a wide size range (2-50 nm). The nanoparticles size can be customized based on intended applications and exemplary particles range in size from 100-450 nm in diameter. The nanoparticles are also made cationic, anionic, or neutral by incorporating selective additives. FIG. 3 (A-E) shows electron microscope images of samples of the exemplary nanoparticles. Each exemplary nanoparticle is exhibits a capability to encapsulate magnetic cores of various sizes (from 2-50 nm in size, e.g. 5 nm, 10 nm, or 20 nm in size) while keeping the final particle size<450 nm. The corresponding designs of exemplary particles are shown in FIGS. 2A through 2G).

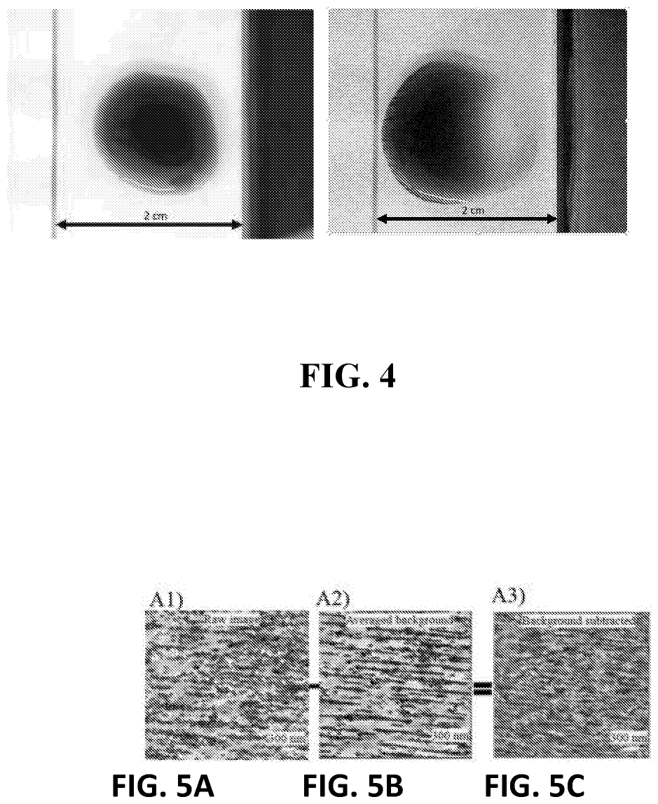

FIG. 4 shows that the exemplary nanoparticles on glass slide in aqueous buffer (1% SDS) and shows that the particles respond to a magnetic gradient. An exemplary instance is shown above.

FIGS. 5A through 5C show the results from image processing to determine particle speed through media. FIG. 5A shows raw snapshot of PLGA MNPs, FIG. 5B shows averaged background of PLGA MNPs, and FIG. 5C shows a snapshot of PLGA MNPs. The MNPs were viewed under an inverted epifluorescence microscope (Zeiss Axiostar plus) using 10× zoom objective optical lens. From images like these, the nanoparticles responded to the magnetic gradient.

### Example 2

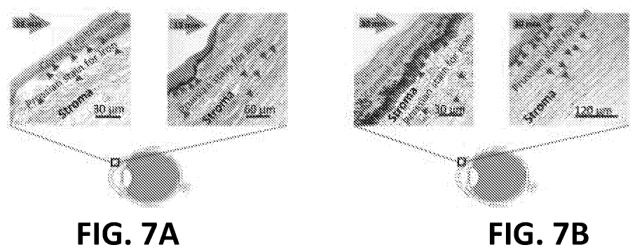

FIGS. 7A and 7B show Prussian staining of iron oxide after delivery into cow eyes and verified that examplary PLGA iron-oxide nano-particles could traverse the epithelial layer of the eye (similar to the epithelial layer of the skin, acts as a barrier) and enter target tissue behind this layer. The quantitative amount of iron-oxide delivered was typically measured by ICP-MS or ICP-OES (inductively coupled plasma mass-spectrometry or optical emission spectrometry) and provided a measure of how many particles were delivered to the target (since the amount of iron-oxide per particle had been previously measured). The amount of therapy delivered to the target could be measured by multiple methods, and in exemplary instances we used HPLC-MS (high performance liquid chromatography mass spectrometry) to measure the amount of drug delivered. This also provided a measure of how many particles were delivered to the target since the amount of therapy per particle had also been previously measured.

The motion of particles through tissue barriers was tested in multiple different live animal studies. In a first set of studies, to-be-tested particles were placed in the outer ear canal of rats, and then a magnetic gradient was applied with a push device to test motion of the particles through the ear drum (the tissue barrier) to the middle ear tissues (the target). In a second set of studies, to-be-tested particles were placed in the middle ear or rats and mice by a syringe, and then a magnetic gradient was applied with a push device to test motion of the particles through the window membranes (the tissue barriers) to the cochlea (the target). In a third set of studies, to-be-tested particles were placed on the surface of the eye of rats and then a magnetic gradient was applied by a pull magnet to test motion of the particles through the sclera (the tissue barries) into the eye and to the retina (the target). In a fourth set of studies, to-be-tested particles were placed on the surface of the skin in rat paws and then a magnetic gradient was applied by a pull magnet to test motion of the particles through the top epithelial layer of the skin (the tissue barries) into underlying skin layers and all the way to the hypodermis (the target).

Tests were also conducted tests in large animal and human cadavers. In a first, second, and third set of cadaver studies, to-be-tested particles were placed in the middle ear of swine, sheep, and cats, and then a magnetic gradient was applied with a push device to test motion of the particles through the window membranes (the tissue barriers) to the cochlea (the target). In a fourth set of studies, particles were also tested for their ability to cross the window membranes and enter the cochlea in human cadaver studies. In a fifth set of studies, to-be-tested particles were placed on the surface of the eye of cows and then a magnetic gradient was applied by a pull magnet to test motion of the particles through the sclera (the tissue barries) and into the eye (the target).

In all cases for live animal and for cadaver studies, whether the iron-oxide nanoparticles did or did not reach their target was determined by extracting the target tissue (after animal sacrifice for live animals), and then measuring the presence and amount of iron-oxide and therapy delivered to the target tissue. The presence of particles in target tissue was qualitatively assessed by Prussian blue staining of tissue. Prussian blue is a stain for iron-oxide and showed if particles had (or had not) reached target tissue.

### Example 3

This example includes a particle having PLGA molecular in a weight range of 30-60 g/mol. This molecular weight range can achieve the required drug release between 7 days and 3 months. PLGA viscosity was about 0.55-0.75 dL/g. The nanoparticles size ranged from about 100 and 500 nm and with therapy release between 10 days and 1 month. PLGA has functional groups: A=Carboxylic (COOH), to achieve faster release of drugs/therapy (<3 months), B=Ester, to develop nanoparticles for long acting release (LAR) system (>3 months), LGA zeta potential: −5 to −30 mV. This attributes efficient colloidal stability of nanoparticles.

The diameter iron-oxide core was about 3-50 nm. Iron-oxide cores within this range exhibit excellent magnetic properties and efficient loading of cores in PLGA nanoparticles is achieved.

Concentration of iron-oxide cores in the PLGA matrix: [Fe]=0.06-0.30 mg (iron)/mg (PLGA). Magnetic-core loading of iron-oxide cores (3-50 nm) is very efficient without adversely affecting the PLGA nano-particle size.

The nanoparticle was composed of Fe₂O₃ and stabilized by oleic acid. A monodispersed, and superparamagnetic iron oxide-cores was desired. The Polydispersity index (PDI) was about 0.01-0.2. Highly monodisperse and homogenous size distribution with little variation from core to core (uniformity).

The iron concentration in cores was about 15% to 20%. To achieve superparamagnetic property and high magnetic content. Magnetic susceptibility: 1×10\hat{ }−5 to 3×10\hat{ }−5. This range ensures maximum encapsulation of iron oxide nano-cores of sizes between 3 and 20 nm.

Magnetic responsiveness: travel with a speed of 50-100 μm/s under a 3 T/m magnetic gradient, in water. The speed range enables PLGA nanoparticles to move effectively through biological barriers.

Polyvinylalcohol (PVA): Mw=31,000-50,000 g/mol, (degree of hydrolyzation: 98-99%). The PVA used produces PLGA nanoparticles in the desired size range 200-280 nm and with release profiles between 7 days and 3 months.

The particle was loaded with drug, proteins, or genes.

### Example 4

This example includes cationic PLGA (poly lactic-co-glycolic acid) nanoparticles, with iron-oxide nano-cores, loaded with therapy in PLGA matrix, stabilized by positively charged phospholipids and surfactant PVA (polyvinyl alcohol), and lyophilized (flash frozen), and gamma or e-beam irradiated for sterility.

Diameter PLGA nanoparticles: 180-280 nm. Because PLGA is biocompatible, biodegradable, and their release profile can easily be tuned by choosing the right molecular weight, compositional ratio (lactide:galactide), density, and functional end groups.

Polydispersity index (PDI): 0.1-0.5. Means the nanoparticles distribution is homogenous and thus no size variance or particle heterogeneity. Little variation from particle to particle.

PLGA molecular weight range: 30-60 g/mol. This molecular weight range is the best to achieve the required drug release between 7 days and 3 months.

PLGA viscosity: 0.55-0.75 dL/g. Best to obtain nanoparticles of desired size range (100-500 nm) and with therapy release between 10 days and 1 month.

PLGA has functional groups:

A=Carboxylic (COOH), to achieve faster release of drugs/therapy (<3 months)

B=Ester, to develop nanoparticles for long acting release (LAR) system (>3 months) PLGA zeta potential: +10 to +30 mV. This attributes efficient colloidal stability of nanoparticles

Cationic lipids: cationic lipids were used to generate positively charged PLGA nanoparticles for enhanced permeation through biological membranes.

Surfactant additives, to enable motion through oily barriers:

DOTAP: 1,2-dioleoyl-3-trimethylammonium-propane (chloride salt)

DOTMA: 1,2-di-O-octadecenyl-3-trimethylammonium propane (chloride salt)

DC-Cholesterol: 3ß-[N-(N′,N′-dimethylaminoethane)-carbamoyl]cholesterol hydrochloride

DOPE: 1,2-Dioleoyl-sn-glycero-3-phosphoethanolamine

Diameter iron-oxide cores: 3-50 nm. Iron-oxide cores within this range exhibit excellent magnetic properties and efficient loading of cores in PLGA nanoparticles is achieved. Concentration of iron-oxide cores in the PLGA matrix:[Fe]=0.06-0.30 mg (iron)/mg (PLGA). Magnetic-core loading of iron-oxide cores (3-50 nm) is very efficient without adversely affecting the PLGA nano-particle size. The nanoparticle is composed of Fe₂O₃ and stabilized by oleic acid. To obtain high quality, monodispersed, and superparamagnetic iron oxide-cores.

The polydispersity index (PDI) is between about 0.01-0.2. Highly monodisperse and homogenous size distribution. The iron concentration in cores was between in 15% to 20% range. To achieve superparamagnetic property and high magnetic content.

Magnetic susceptibility: 1×10̂−5 to 3×10̂−5. This range ensures maximum encapsulation of iron oxide nano-cores of sizes between 3 and 20 nm.

Magnetic responsiveness: The nanoparticle travels with a speed of 50-100 μm/s under a 3 T/m magnetic gradient, in water. The speed range enables PLGA nanoparticles to move effectively through biological barriers.

Polyvinylalcohol (PVA): Mw=31,000-50,000 g/mol, (degree of hydrolyzation: 98-99%). The PVA used produces PLGA nanoparticles in the desired size range 200-280 nm and with release profiles between 7 days and 3 months.

Therapy: Particle can be loaded with drug, proteins, or genes.

### Example 5

This example includes nanoparticles that are a blend of PLGA (poly lactic-co-glycolic acid)+polymethacrylate-based copolymers (Eudragit, RLPO), with iron-oxide nano-cores, loaded with therapy in PLGA matrix, stabilized by surfactant PVA (polyvinyl alcohol), and lyophilized (flash frozen), and gamma or e-beam irradiated for sterility.

Diameter PLGA nanoparticles: 160-250 nm. Because PLGA is biocompatible, biodegradable, and their release profile can be tuned by choosing the right molecular weight, compositional ratio (lactide:galactide), density, and functional end groups.

Eudragit (RL PO), copolymers of ethyl acrylate, methyl methacrylate and a low content of methacrylic acid ester with quaternary ammonium groups with a molecular weight of 32,000 g/mol.

RL PO was used in combination with PLGA to attain,

Positively charged nanoparticles. Positive charge allows better motion through tissue barriers.

Customised release of therapy. Allows release of therapy at a desired rate.

The polydispersity index (PDI) was about 0.1-0.5. The nanoparticles distribution is homogenous between this range and indicates no size variance or particle heterogeneity.

The PLGA molecular weight range was 30-60 g/mol. The molecular weight range is best to achieve the required drug release between 7 days and 3 months.

PLGA viscosity: 0.55-0.75 dL/g. The nanoparticles had a size ranging from about 100-500 nm and with therapy release profile between 10 days and 1 month. The Diameter iron-oxide cores was about 3-50 nm. Iron-oxide cores within this range exhibit excellent magnetic properties and efficient loading of cores in PLGA nanoparticles is achieved.

Concentration of iron-oxide cores in the PLGA matrix: [Fe]=0.06-0.30 mg (iron)/mg (PLGA). Magnetic-core loading of iron-oxide cores (3-50 nm) is very efficient without adversely affecting the PLGA nano-particle size.

Composed of Fe₂O₃ and stabilized by oleic acid. To obtain high quality, monodispersed, and superparamagnetic iron oxide-cores.

Polydispersity index (PDI): 0.01-0.2. Highly monodisperse and homogenous size distribution. Little variation from core to core.

Iron concentration in cores: The iron (Fe) in 15% to 20% range. To achieve superparamagnetic property and high magnetic content.

Magnetic susceptibility: 1×10̂−5 to 3×10̂−5. This range ensures maximum encapsulation of iron oxide nano-cores of sizes between 3 and 20 nm.

Magnetic responsiveness: travel with a speed of 50-100 μm/s under a 3 T/m magnetic gradient, in water. The speed range enables PLGA nanoparticles to move effectively through biological barriers.

Polyvinylalcohol (PVA): Mw=31,000-50,000 g/mol, (degree of hydrolyzation: 98-99%). The PVA used produces PLGA nanoparticles in the desired size range 200-280 nm and with release profiles between 7 days and 3 months.

The particle can be loaded with drug, proteins, or genes.

### Example 6

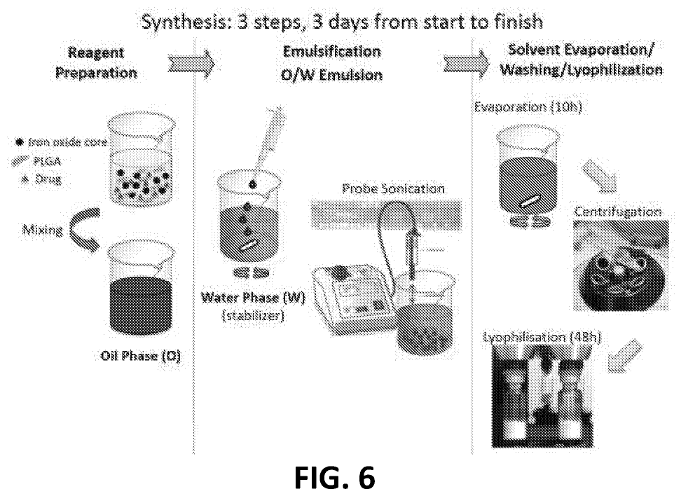

FIG. 6 shows a schematic view of a manufacturing process of an exemplary nanoparticle includes the following steps.

Step 1: The cationic polymeric nanoparticles are formulated using a biodegradable poly (D,L-lactide-co-glycolide) (PLGA) polymer matrix containing magnetic iron oxide cores, cationic lipid surfactant DOTAP (1,2-dioleoyl-3-trimethylammonium-propane (chloride salt), and the drug (prednisolone acetate, PSA) using a single emulsion solvent evaporation (SESE) procedure.

Step 1a. In a typical procedure 10 mg of PSA (Prednisolone 21-acetate) is dissolved in 5 ml of chloroform (CHL) by intermittent cycles of vortex and incubation in a warm water bath maintained at 37 C.

Step 1b. Once a clear drug solution is obtained, 12.5 mg of DOTAP (1,2-dioleoyl-3-trimethylammonium-propane (chloride salt), Avanti Biolipids) is added followed by 50 mg of PLGA (lactide:glycolide (50:50) 30,000-60,000 Da) at room temperature. The organic phase is vigorously mixed to ensure all ingredients are dissolved and a clear solution is obtained. Finally, 800 μl of magnetic cores are added to the obtained organic phase.

Step 1c. The organic phase is vortexed and sonicated in a water bath for 10 seconds in pulses.

Step 1d. The obtained organic phase is dropped into 50 ml of PVA solution (2% polyvinyl alcohol, 31,000-60,000 Da) under continuous magnetic stirring and subjected to probe sonication for 5 min in an ice/water bath.

Step 1e. The smooth-milky emulsion obtained above is left to stir on a magnetic stir plate for 18 hours to ensure complete evaporation of the organic solvent.

Step 2: The above obtained nanoparticle emulsion is split into two 50 ml falcon centrifuge tubes and centrifuged at 12000 rpm for 60 min to collect the nanoparticle pellet. The pellet is re-dispersed (vortex-sonication cycles) in 15 ml deionized water and is centrifuged as above. The centrifugation process is repeated twice to remove any free and excess of reagents. The resulting pellet is then freeze-dried using a tower lyophilizer as described next.

Step 3: Lyophilization: In a typical procedure the nanoparticles pellet obtained above is hen re-dispersed in a glass vial containing 3 ml of sugar solution (2% Trehalose). The sample is then frozen (−80 C for 24 hours (or) Flash frozen in liquid nitrogen N₂ for 3 min) before placing in a lyophilizer for 48 hours. The final product obtained is a fine free-flowing powder.

### Example 7

### Drug Loading Method

(A) Co-Loading of Drug Combinations (Steroids and Antibiotics) into Magnetic PLGA Nanoparticles

The Surfactant types : sodiumdodecylsulfate (SDS), docusate sodium (Doc Na), sodium deoxycholate (Na DeOxyChol), dextran sulfate (DS), etc.

Antibiotics: Ciprofloxacin/Ciprofloxacin hydrochloride (CIP.HCl), Levofloxacin (LVFX), Ofloxacin (OFLX), etc.

Steroids: Prednisolone 21 acetate (PSA), Dexamethasone 21 acetate (DexA), Fluocinolone acetonide (FA), Dexamethasone (Dex), Prednisolone (PS), etc.

One step: In-situ formation of Hydrophobic Ion Pairing (HIP) complex between antibiotic and surfactant/s followed by co-loading with steroid. Fluocinolone acetonide (FA) was dissolved in DCM by intermittent cycles of vortex and incubation in a water bath maintained at 37 C. 100 mg of PLGA-COOH followed by 600 ul of iron oxide cores was dissolved in the above obtained oil phase (O).

5 mg of CIP.HCl was dissolved in 0.5 ml of water and incubated at 37 C (water bath) for 10 minutes to ensure complete solubility. This is called water phase (W1.1)

25 mg of DS was dissolved in 0.5 ml of water and vortexed. This is called water phase (W1.2)

W1.1 was mixed with (O) phase and subjected to probe sonication at 30% amplitude for 1 minute (⅛″ solid probe, QSonica Q500, 500 watts, 20 kHz) in an ice/water bath. This resulted in W1.1/O emulsion.

To the above W1.1/O emulsion was added 0.5 ml of water phase W1.2 followed by probe sonication at 30% amplitude for 1 minute (⅛″ solid probe, QSonica Q500, 500 watts, 20 kHz) in an ice/water bath. This resulted in W1.1/O/W1.2 emulsion.

To the above W1.1/O/W1.2 emulsion was added 5 ml of 1% PVA (W2) followed by vortexing for 20 sec. The mixture was then subjected to probe sonication at 30% amplitude for 3 minutes (⅛″ solid probe, QSonica Q500, 500 watts, 20 kHz) in an ice/water bath. This resulted in (W1.1/O/W1.2)/W2 emulsion.

The emulsion from above was diluted with 40 ml of 1% PVA and was transferred into a 100 ml beaker. The diluted emulsion was left to stir on a magnetic stir plate for 4 hours to ensure complete evaporation of the organic solvent and resulting in the formation of polymer nanoparticles.

The nanoparticles solution obtained above was split into two 50 ml falcon centrifuge tubes and was centrifuged at 13500 rpm for 30 minutes to collect the nanoparticle pellet. The pellet was redispersed (vortex-sonication cycles) in 15 ml water and was centrifuged as above. The centrifugation process was repeated twice to remove any free and excess of reagents. The resulting pellet was freeze-dried using a tower lyophilizer as described below.

Lyophilization: In a typical procedure the nanoparticle pellet obtained above was re-dispersed in 3 ml of sugar solution (2% Trehalose) and transferred into a 20 ml glass vial. The nanoparticle-sugar suspension was flash frozen in liquid N₂ for 2 minutes and lyophilized for 48 hours. The final product obtained was a fine free-flowing powder.

(II) Two step: Pre-formation of HIP complex between antibiotic and surfactant/s followed by co-loading with steroid

5 mg of CIP.HCl was dissolved in 0.5 ml of water and incubated at 37 C (water bath) for 10 minutes to ensure complete solubility.

3 mg of DS was dissolved in 0.5 ml of water and vortexed.

0.5 ml of CIP.HCl solution was dropwise introduced into 0.5 ml of DS, followed by vortexing the mix.

The mixture was then allowed to mix on a rocker for 10 minutes at room temperature before centrifuging for 5 minutes at 14000 rpm.

The resultant pellet of CIP-DS HIP complex (S) was redispersed in water by vortexing and was centrifuged resulting in a pellet. The washing step was repeated twice.

The complex was dried in a vacuum centrifuge at 30 C for 4 hours resulting in a dry pellet also called as the solid (S) phase.

5 mg of Fluocinolone acetonide (FA) was dissolved in 2 ml of DCM by intermittent cycles of vortex and incubation in a water bath maintained at 37 C. This is known as the oil phase (O). 100 mg of PLGA-COOH followed by 600 ul of iron oxide cores was dissolved in the above obtained oil phase (O).

The CIP-DS complex (S) was redispersed in the FA+PLGA-COOH solution (O) and vortexed for 20 sec. The mixture was then subjected to probe sonication at 30% amplitude for 1 minutes (⅛″ solid probe, QSonica Q500, 500 watts, 20 kHz) in an ice/water bath. This resulted in S/O emulsion. To the above S/O emulsion was added 5 ml of 1% PVA (W) followed by vortexing for 20 sec. The mixture was then subjected to probe sonication at 30% amplitude for 3 minutes (⅛″ solid probe, QSonica Q500, 500 watts, 20 kHz) in an ice/water bath. This results in S/O/W emulsion. The S/O/W emulsion from above was diluted with 25 ml of 1% PVA and was transferred into a 100 ml beaker. The diluted emulsion was left to stir on a magnetic stir plate for 4 hours to ensure complete evaporation of the organic solvent and resulting in the formation of polymer nanoparticles.

The nanoparticles solution obtained above was split into two 50 ml falcon centrifuge tubes and was centrifuged at 13500 rpm for 30 minutes to collect the nanoparticle pellet. The pellet was redispersed (vortex-sonication cycles) in 15 ml Water and was centrifuged as above. The centrifugation process was repeated twice to remove any free and excess of reagents. The resulting pellet was freeze-dried using a tower lyophilizer as described below.

Lyophilization: In a typical procedure the nanoparticle pellet obtained above was re-dispersed in 3 ml of sugar solution (2% Trehalose) and transferred into a 20 ml glass vial. The nanoparticle-sugar suspension was flash frozen in liquid N₂ for 2 minutes and lyophilized for 48 hours. The final product obtained was a fine free-flowing powder.

(B) Polymer Coated Magnetic Nanoparticles Loaded with Drugs (Steroids and Antibiotics)

Oleic acid stabilized magnetic iron oxide cores (10, 20, 30 nm) were synthesized in house. 10 mg of steroid was dissolved in 5 ml of Chloroform and was mixed with iron oxide nanoparticles.

The nanoparticle-drug solution was left to mix at room temperature for 3-5 hours and was magnetically separated and washed with ethanol to remove any free drug molecules. The resulting steroid-iron oxide complex was redispersed in hexane.

Block copolymers Pluronics (F68, F127, etc.) were used to stabilize the above obtained steroid-iron oxide complex. Different amounts of block copolymers were dissolved in PBS buffer and was mixed with equal volumes of hexane containing drug-iron oxide complex. The above reaction mixture was allowed to mix at 30 C for 12 hours and was washed twice with hexane:water (1:1).

A complete phase transfer of iron oxide cores from organic solvent to aqueous phase was achieved post functionalization of Pluronic polymers.

### Example 8

In an exemplary process to measure drug release from exemplary particles, a stock solution (1 mg/ml) of lyophilized particles was placed in artificial cerebrose spinal fluid (aCSF, pH 7.4) and transferred immediately to glass vials in equal volumes (1 ml). The samples were then placed in a shaker/incubator at a constant temperature of 37° C. At exemplary time intervals (e.g. at 0, 0.5, 1, 4, 9, 24, 48 and 72 hours) formulation vials (e.g. in duplicate: n=2) were withdrawn and centrifuged at 18,000 g for 10 min. The supernatant solution was separated from the pellet and mixed with an equal volume of acetonitrile for HPLC analysis. Exemplary particles have been designed and synthesized to have fast release of therapy (within minutes or hours), or for slow release of therapy (within weeks or months).

The foregoing description of several methods and embodiments has been presented for purposes of illustration. It is not intended to be exhaustive or to limit the claims to the precise steps and/or forms disclosed, and obviously many modifications and variations are possible in light of the above teaching.

1. A nanoparticle capable of crossing tissue, comprising
an iron oxide core,
a first therapeutic agent, and
a polymeric coating, wherein the coating degrades in water at about 37 degrees.

2. The nanoparticle of claim 1, wherein the core is between 3 and 30 nanometers in diameter.

3. The nanoparticle of claim 1, wherein the core is between 10 and 100 nanometers in diameter.

4. The nanoparticle of claim 1, wherein the coating is PLGA

5. The nanoparticle of claim 1, wherein the coating is a Poloxmer coating.

6. The nanoparticle of claim 1, further comprising a second therapeutic agent.

7. The nanoparticle of claim 1, wherein the first therapeutic agent is ciprofloxacin.

8. The nanoparticle of claim 1, wherein the first therapeutic agent is fluocinolone acetonide.

9. The nanoparticle of claim 1, wherein the first therapeutic agent is dexamethasone.

10. A method for treating a patient, comprising:
providing a lyophilized composition of nanoparticles,
reconstituting the nanoparticles,
applying the nanoparticles to a site, and
moving the nanoparticles to a target site using a magnetic gradient.

11. A method for providing a therapeutic agent to a subject comprising administering to a subject in need thereof a magnetic nanoparticle containing a first therapeutic agent and a magnetic core, and moving the nanoparticles to a target site using a magnetic gradient.

12. The method of claim 1, further comprising directing the particle within the subject using a magnet.

13. A composition of nanoparticles, wherein the nanoparticles are lyophilized.

14. A method for preparing a nanoparticle composition having a first agent and a second agent, comprising
a. Forming a hydrophobic ion complex between the first agent
b. Adding the second agent after the formation of the hydrophobic ion complex.

15. A lyophilized pharmaceutical composition, comprising
a. a nanoparticle capable of crosses tissue having an iron oxide core, a first therapeutic agent, and a polymeric coating, wherein the coating degrades in water at about 37 degrees.
b. a sugar around the nanoparticle.

16. A nanoparticle comprising an emulsified polymer, a surfactant, a magnetic core, a first biologically active agent, and a second biologically active agent, wherein the first biologically active agent is complexed to form a hydrophobic ion complex.

17. A method of claim 14, further comprising lyophilizing the composition.

18. A method of claim 14, further comprising sterilizing the composition using radiation.
